(19) United States
(12) Patent Application Publication
(10) Pub. No.: US 2020/0029994 A1
(43) Pub. Date: Jan. 30, 2020

(54) SURGICAL BLADE HANDLE ASSEMBLY AND METHOD FOR USE

(71) Applicant: Progressive Medical, Inc., Fenton, MO (US)

(72) Inventor: Mark M. Martin, Fenton, MO (US)

(21) Appl. No.: 16/509,024

(22) Filed: Jul. 11, 2019

Related U.S. Application Data
(60) Provisional application No. 62/703,186, filed on Jul. 25, 2018.

(51) Int. Cl. A61B 17/3211; A61B 17/00

(52) U.S. Cl. CPC A61B 17/3211; A61B 2017/0046; A61B 2017/00477; A61B 17/00234

(57) ABSTRACT

Herein disclosed is a surgical blade handle assembly and methods of use.

### CROSS REFERENCE TO RELATED APPLICATIONS

The present application claims priority to U.S. provisional application Ser. No.. 62/703,186, filed Jul. 25, 2018, herein incorporated by reference in its entirety.

### FIELD OF THE INVENTION

The present invention relates generally to a laparoscopic surgical device. More particularly, the present invention relates to a laparoscopic scalpel that facilitates enlargement of a laparoscopic incision through the peritoneum, fascia, and abdominal wall muscles for extraction of solid organs or other specimens, and/or introduction of intraluminal staplers or other devices.

### BACKGROUND OF THE INVENTION

Laparoscopic surgery, also known as “minimally invasive” surgery, is a method for performing surgery via one or more small incisions typically on the order of about 1 cm across. It is known that laparoscopic surgery offers many benefits to patients and the healthcare system including, for example, less pain, shorter hospital stay, quicker return to normal activity and to work, and better cosmetic results. These benefits make the health care system more efficient; thus laparoscopic techniques are in high demand.

Laparoscopic surgery has revolutionized patient care in the last 20 years. In many cases, minimally invasive approaches have become the standard care, even for specialties such as gynecology, urology, kidney transplant, and foregut surgery. In 2010, there were 2.8 million minimally invasive procedures performed in the United States alone, representing an increase of about 2.6% from 2009. However, these procedures include only about 38% of colectomies and 25% hysterectomies being performed in a minimally invasive fashion. Furthermore, these procedures do not include the usage of the current inventive device in procedures such as adrenalectomy, gastrectomy, donor and pathological nephrectomies, prostates, and liver resections. Therefore, the number of minimally invasive procedures is expected to continue to rise as technology expands.

Furthermore, with the advent of ‘robotic’ surgery more and more techniques are being created. In the past, many surgeons abstained from laparoscopic or robotic techniques because it was felt to not offer an advantage if solid organs required extraction. For example, if a surgeon was doing a colon resection, it was not uncommon for surgeons to perform the operation in an ‘open’ fashion or quickly convert from laparoscopic to open knowing that they were going to require an open incision to extract the solid organ in question.

As more intra-abdominal surgery is being offered with minimally invasive techniques, surgeons need to have the ability to extract a specimen, for example a solid organ, either whole or morcellated. To facilitate the removal of larger solid organs such as, for example, kidneys, adrenals, the uterus, colon, small bowel, gallbladder, and tumors, a surgeon has to create a sufficiently wide extraction site on the peritoneal surface of the abdominal wall. However, in order to preserve the integrity of the minimally invasive operation, surgeons strive to remove the specimen through the smallest possible skin incision. Therefore, a problem may arise when a specimen needing to be removed is larger than any of the laparoscopic incisions.

Currently, there is no reliable ‘sharp’ option available for lengthening a laparoscopic incision. For example, if an organ requires extraction, the skin is cut with a scalpel; however, the fascia, muscle, and peritoneum are all bluntly ‘ripped’ and spread apart by the surgeon's fingers or a blunt instrument to allow for extraction of the organ. This blunt method, while adequate, is imprecise, takes increased time to execute, and leads to a considerable amount of tissue trauma, causing increased postoperative pain. In addition, this blunt method leads to the instant loss of the pneumoperitoneum, which is gas normally sealed within the body cavity. Such loss of the pneumoperitoneum may result in a massive expulsion of carbon dioxide gas and aerosolized body fluid when the organ is removed under pressure of the pneumoperitoneum. Such expulsion may expose the surgeons and operating room technologists to an infectious disease hazard and therefore is a safety issue.

Although not widely used, a “fascia scalpel” manufactured by LiNA Medical in Glostrup, Denmark, is designed to be inserted next to a specimen bag which is being pulled up against the abdominal wall. This creates problems as the bag will tend to surround and engulf the LiNA scalpel, and the specimen bag can be cut which can lead to loss of integrity of the bag, loss of containment of the specimen, and potential dissemination of contaminated fluid, for example, an infected appendix, or cancerous cells, for example, a malignant tumor. Thus, the LiNA scalpel is designed for cutting more superficially on the abdominal wall or the more dorsal part of the skin and is not designed for the more ventral part of the abdominal wall. The LiNA fascia scalpel is designed for use with smaller solid organs like the appendix and/or gallbladder. The LiNA fascia scalpel is not designed to be placed through a trocar, nor does the LiNA scalpel have a blunt distal end to protect tissue from unintended damage. Additionally, the LiNA fascia scalpel does not enable surgeons to use the instrument under visualization nor does it allow surgeons to work without releasing the pneumoperitoneum.

Further issues exist with using a traditional surgical scalpel to enlarge a laparoscopic incision. For example, when using a regular surgical scalpel attached to a standard or longer handle, it is possible that the scalpel blade may be dislodged from the handle by the abdominal wall tissue and/or the rubber housing of existing trocars if the scalpel is introduced inside the trocar. In addition, standard cutting with a scalpel is not protected and surrounding tissues can be unintentionally injured. Further, a traditional surgical scalpel or a LiNA fascia scalpel can slip and fall freely into the abdomen creating an enterotomy (bowel injury) and thus have potentially devastating consequences.

A need exists for a laparoscopic scalpel that can precisely enlarge a laparoscopic incision without compromising the pneumoperitoneum. Such a device would safely, quickly, and efficiently incise the peritoneum, fascia, and abdominal wall muscles to extract solid organs or introduce intraluminal staplers or other devices. Such a device would allow for extraction of larger specimens, which allows for wider applicability across multiple specialties including, but not limited to urology, gynecology, general surgery, bariatric surgery, endocrine surgery, colo-rectal surgery, liver surgery, and thoracoscopic surgery of the chest.

The present invention attempts to solve these problems as well as others.

### SUMMARY OF THE INVENTION

Herein disclosed is a surgical blade handle assembly and methods of use. A surgical blade handle assembly generally comprises: a handle operably coupled with a button driver and a proximal end of a shaft; a blade disposed on the distal end of the shaft and the blade operably coupled with the button driver to protrude out of the inner pocket of the shaft; the surgical blade handle assembly includes a horizontal axis and a vertical axis, where the button driver operates to displace the blade along the vertical and horizontal axis; the button driver operates from a first position to a second position as to displace the blade and protrude the blade from the outer surface of the shaft; and the shaft operates to conceal the blade from the outer portion of the shaft and to not cut any tissue once the blade is in the first position; wherein the second position of the blade is in a cutting position and protrudes out of the outer portion of the shaft.

A method of operating a surgical blade handle assembly is disclosed and comprises: coupling a handle operably with a button driver and a proximal end of a shaft, and the surgical blade handle assembly includes a horizontal axis and a vertical axis; disposing a blade on the distal end of the shaft and operably coupling the blade with the button driver to protrude out of the inner pocket of the shaft; operating the button driver to displace the blade along the vertical and horizontal axis; operating the button driver from a first position to a second position as to displace the blade and protrude the blade from the outer surface of the shaft; and operating the shaft to conceal the blade from the outer portion of the shaft and to not cut any tissue once the blade is in the first position; wherein the second position of the blade is in a cutting position and protrudes out of the outer portion of the shaft.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### BRIEF DESCRIPTION OF THE DRAWINGS

In the accompanying figures, like elements are identified by like reference numerals among the several preferred embodiments of the present invention.

FIG. 1A is a perspective view of the blade handle assembly; FIG. 1B is an exploded view of the bland handle assembly.

FIG. 2A is a side view of the button driver; FIG. 2B is a top view of the button driver; FIG. 2C is an enlarged view of the distal end of the button driver; and FIG. 2D is an enlarged view from section 2C in FIG. 2A.

FIG. 3A is a bottom perspective of the blade holder; FIG. 3B is a side perspective view of the blade holder; FIG. 3C is a side view of the blade holder; FIG. 3D is a top view of the blade holder.

FIG. 4A is a cross section of the inner pocket of the shaft; and FIG. 4B is a down view of the inner pocket of the shaft.

FIG. 5A is a cross section of the button driver coupled with the blade holder disposed within the inner pocket; FIG. 5B is a cross section of the button driver coupled with the blade holder and the blade disposed within the inner pocket.

FIG. 6A is a cross section of the button driver coupled with the blade holder and the blade disposed within the inner pocket with the shaft shown in phantom; FIG. 6B is a cross section of the button driver coupled with the blade holder and the blade disposed within the inner pocket.

FIG. 7A is a perspective view of the blade; and FIG. 7B is a side view of the blade.

FIG. 8A is a side view of the blade with the blade holder; FIG. 8B is a side view of the blade with the blade holder; and FIG. 8C is a top view of the blade with the blade holder.

FIG. 9A is a side view of the button driver with the blade holder; and FIG. 9B is a side view of the button driver with the blade holder and the blade.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein. The words proximal and distal are applied herein to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

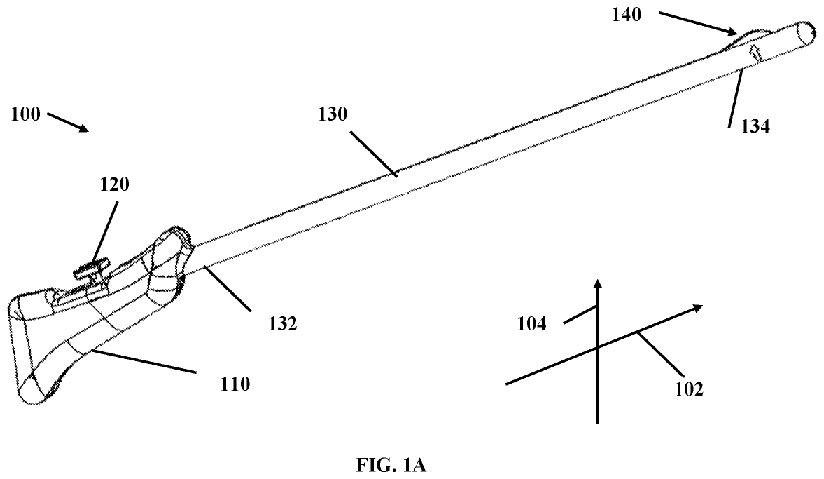

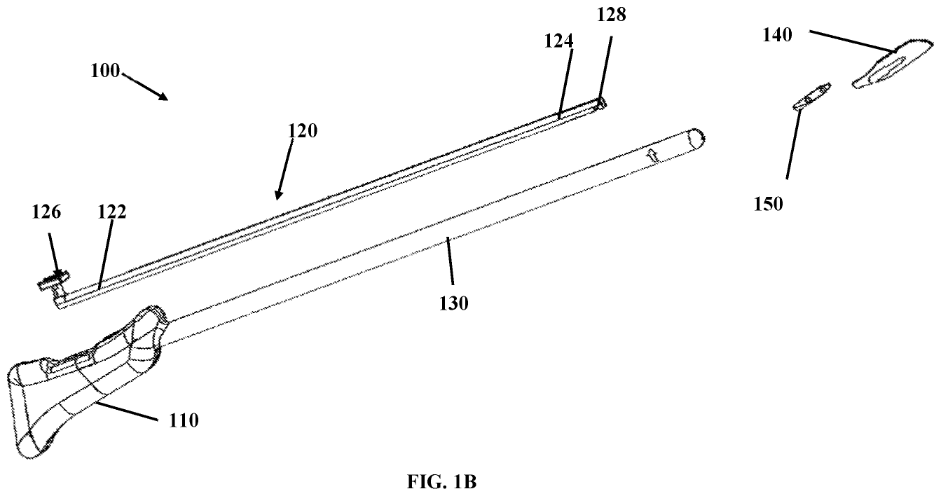

Generally speaking, the surgical blade handle assembly 100 is shown in FIG. 1A and surgical blade handle assembly 100 comprises a handle 110 operably coupled with a button driver 120 and a proximal end 132 of a shaft 130. A blade 140 is disposed on the distal end 134 of the shaft 130 and the blade 140 is operably coupled with the button driver 120 to protrude out of the inner pocket of the shaft 130. The surgical blade handle assembly 100 includes a horizontal axis 102 and a vertical axis 104, where the button driver 120 operates to displace the blade along the vertical 104 and horizontal axis 102. The button driver 120 operates from a first position to a second position as to displace the blade and protrude the blade from the outer surface of the shaft 130. The shaft 130 operates to conceal the blade from the outer portion of the shaft and to not cut any tissue once the blade 140 is in a first position. The second position of the blade is shown in FIG. 1A, where the blade 140 is in a cutting position and protrudes out of the outer portion of the shaft 130.

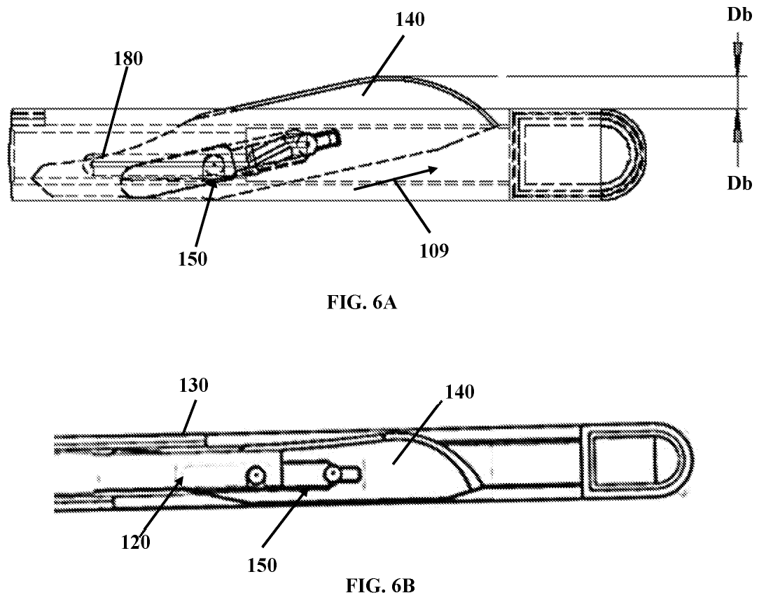

As shown in FIG. 1B, the button driver 120 comprises a proximal end 122 and a distal end 124; wherein the proximal end 122 includes a raised button portion 126 that protrudes out of the handle 110. The distal end 124 includes a holding portion 128 to secure a blade holder 150. The blade holder 150 secures the blade 140 to the button driver 120 and is operably coupled with the shaft 130 to displace the blade 140 within a lumen of the shaft and the outer portion of the shaft. The blade 140 is displaced or protrudes outside the outer portion of the shaft 130 by a distance Db, as shown in FIG. 6A.

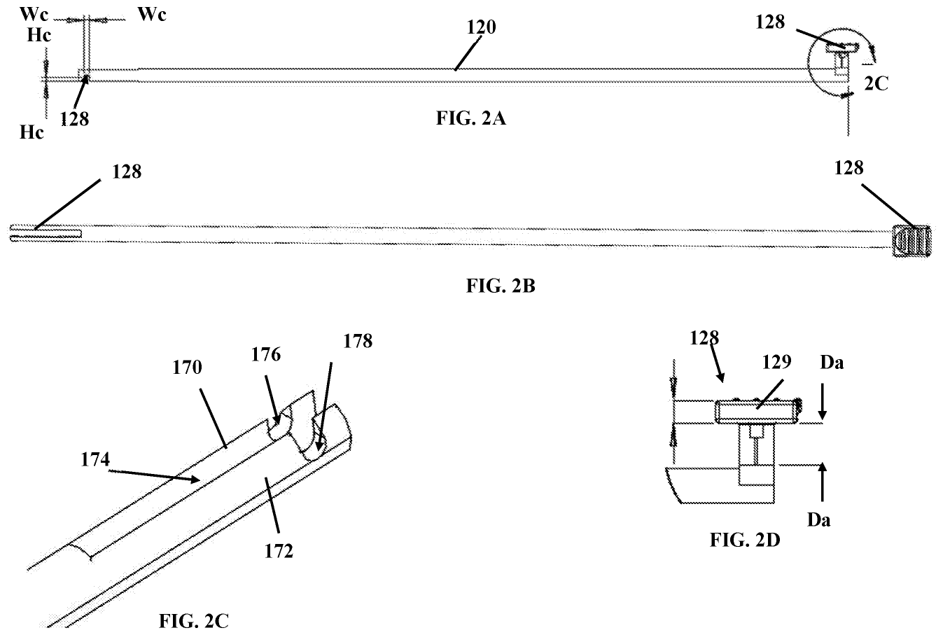

As shown in FIGS. 2A-2C, the holding portion 128 includes a first arm 170 and a second arm 172 with a first slotted portion 174 disposed between the first arm 170 and the second arm 172. As shown in FIG. 2C, the first arm 170 includes a first C-shaped receiver end 176 and the second arm 172 includes a second C-shaped receiver end 178. As shown in FIG. 2D, the button driver includes a button portion 128 that includes a raised feature 129 protruding vertically out of the handle at a distance Da. The button includes a grated surface to enhance gripping by the user or operator, as shown in FIG. 2B. The first and second C-shaped receiver ends 176 and 178 include a width We and a height Hc that receives the holding arms of the blade holder 150 and allows the holding arms of the blade holder 150 to rotate about an axis.

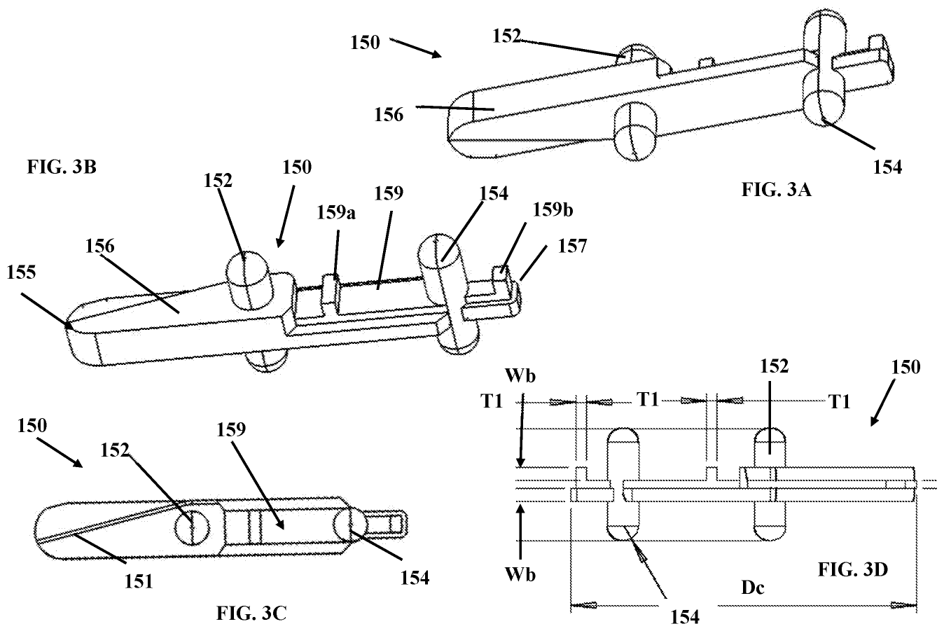

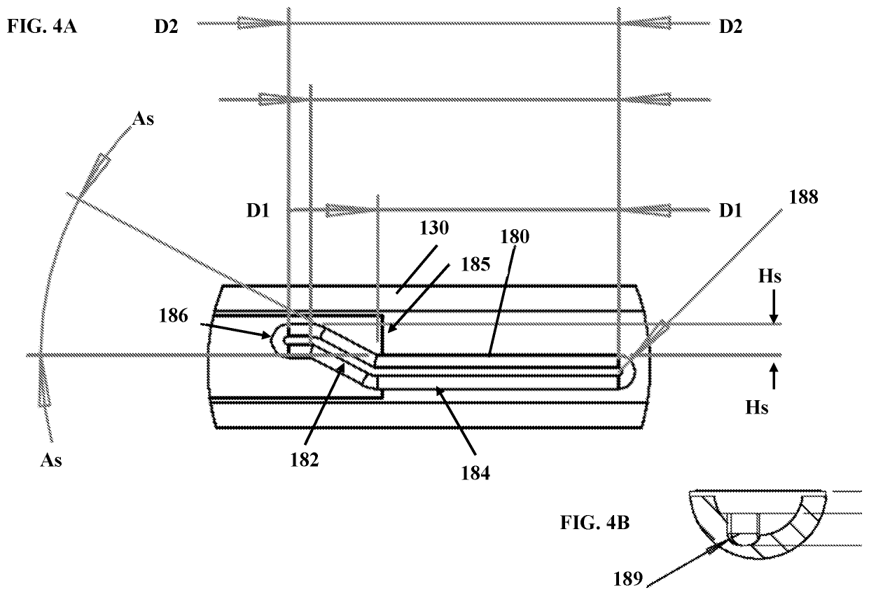

As shown in FIGS. 3A-3C, the blade holder 150 includes a first receiving arm 152 and a second receiving arm 154, wherein the first and second receiving arms 152 and 152 protrude from an inner section 156 of the blade holder. The inner section 156 includes a first end 155 and a second end 157 separated by a distance Db, as shown in FIG. 3D. The inner section 156 includes a stepped portion 159 that includes rectangular profile as to secure a portion of the blade. The inner section 156 also includes a distal slopped portion 151 to permit the blade holder 150 to rotate along the slopped edge of the inner pocket. The stepped portion 159 includes two tabs 159a and 159b as to lock the blade holder into the first position and the second position. The inner section 156 includes a width Wb and the tabs 159a, 159b include a thickness T1 that the less than the width Wb of the inner section 156. The first and second C-shaped receiver ends 176 and 178 receive and hold the first receiving arm 152 and second receiving arm 154 of the blade holder 150 and allow the blade holder 150 to be displaced along the vertical axis of the surgical blade handle assembly. The second receiving arm 154 is operably coupled with the inner pocket 180 of the shaft 130, as shown in FIG. 4A.

As shown in FIG. 4A, the inner pocket 180 of the shaft includes a slopped edge 182 connecting a flat edge 184. The slopped edge 182 includes a second stop 186 and the flat edge includes a first stop 188; and the slopped edge 182 includes a third stop 185. The second stop 186 abuts the second end 157 of the inner section 156 of the blade holder 150 as to prevent the blade holder 150 from moving any further along the vertical or horizontal axis. The third stop 185 abuts with the tabs 159a of the stepped portion 159 as to prevent the blade holder from moving any further along the slopped edge 182. The slopped edge 182 operably couples with the second receiving arm 154 of the blade holder 150 to displace the blade holder 150 along the vertical axis upon proximal displacement of the blade holder 150 along the horizontal axis. The first stop 188 abuts the first end of the 155 of the blade holder to prevent further distal movement of the blade holder along the horizontal axis. The slopped edge 182 includes an angle As and the slopped edge 182 includes a height Hs, where the height Hs is the limiting distance the blade holder 150 and blade is able to be displaced vertically along the vertical axis by way of the second receiving arm 154.

The inner pocket 180 includes a distance D1 that accommodates the blade holder in the first position and the distance Dc of the blade holder. The inner pocket 180 includes a distance D2 that is greater than D1 and Dc to permit the horizontal displacement of the blade holder within the inner pocket 180. The inner pocket 180 includes a lower grooved portion 189 to allow the smooth horizontal displacement of the blade holder within the inner pocket, as shown in FIG. 4B.

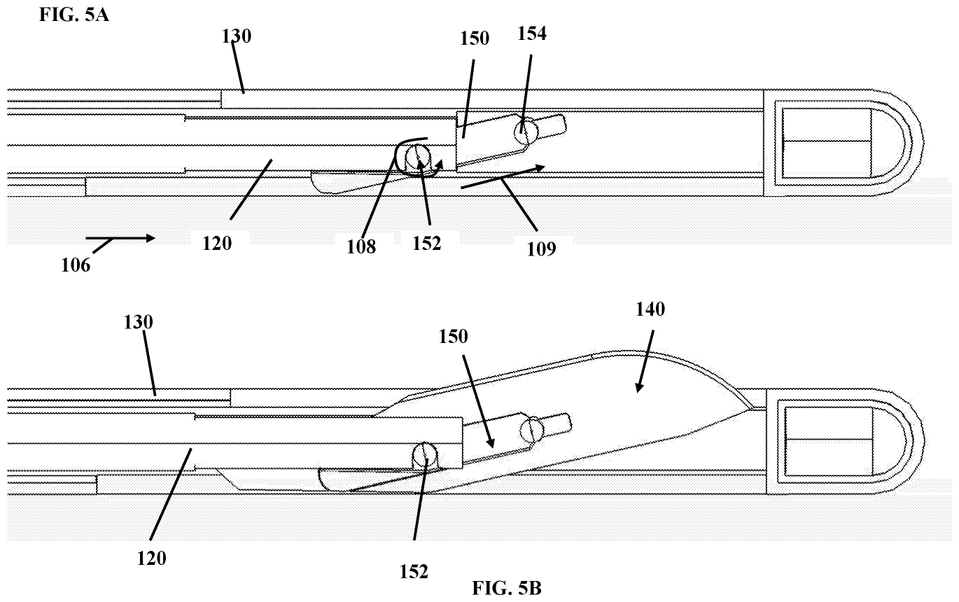

The second position is shown in FIG. 5A, where the button driver 120 is displaced proximally along the longitudinal axis shown by arrow 106 and moving the blade holder 150 and while the blade holder is proximally displaced, the second receiving arm 154 moves at the Angle A and vertically displaces the blade holder and blade. The first receiving arm 152 allows the blade holder to rotate along the vertical axis, generally shown by arrow 108. The second receiving arm 154 moves along the angled edge of the inner pocket 180 (as shown in FIG. 6A) generally shown by arrow 109. The first position is shown in FIG. 6B, where the blade is disposed within the lumen of the shaft and concealed within the shaft,

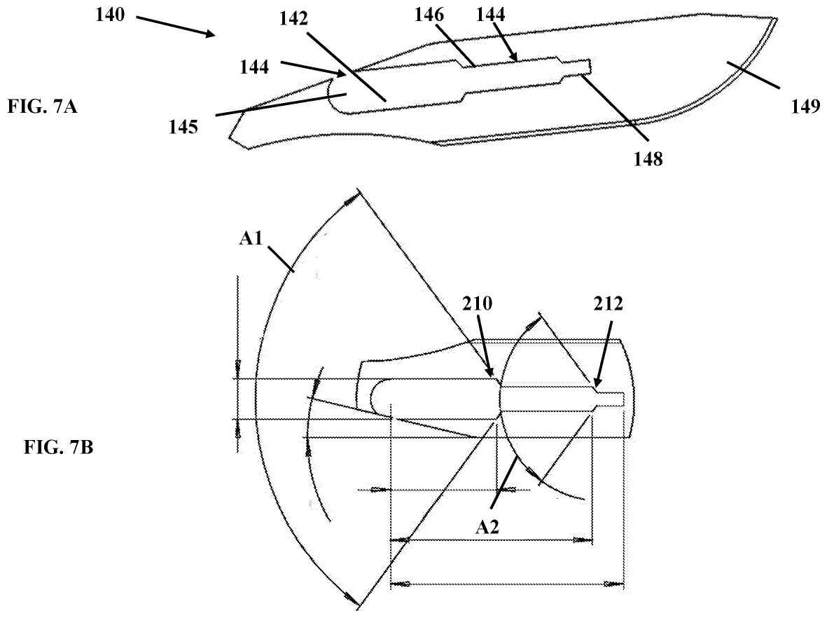

As shown in FIG. 7A, the blade 140 includes an inner cutout 142 configurable to fit over the at least a portion of the inner section of the blade holder. The inner cutout includes an opening 144, a circular end portion 145, a stepped middle portion 146, and a second end cap 148. As such, the stepped portion 159 includes a complementary and reciprocal circular end portion, a stepped middle portion, and a second end cap as to receive the inner cutout 142 of the blade. The blade 140 includes a sharpened portion 149.

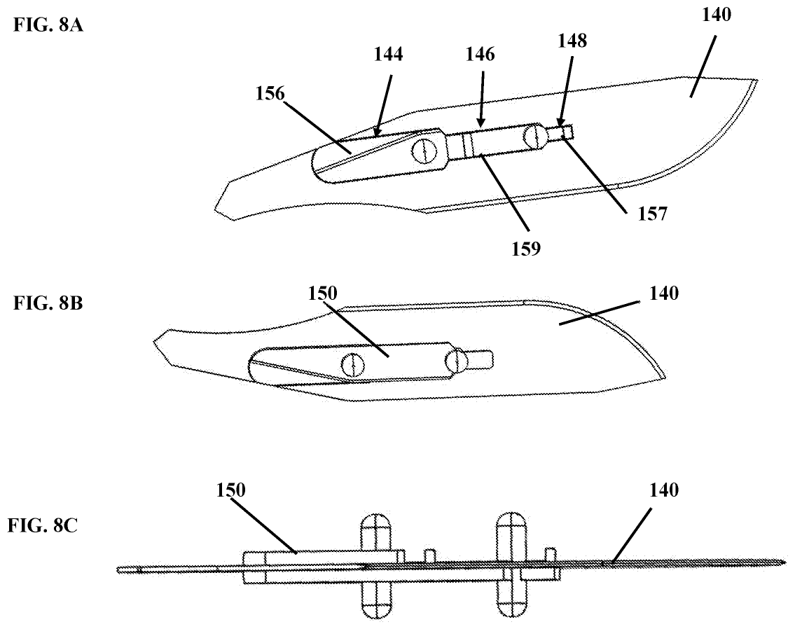

As shown in FIG. 7B, the inner cutout incudes a first lipped portion 210 between circular end portion 144 and the stepped middle portion 146; and a second lipped portion 212 between the stepped middle portion 146 and the second end cap 148. The first lipped portion 210 includes an angle A1 and the second lipped portion 212 includes an angle A1. The first and second lipped portions 210 and 212 assist in securing the blade to the blade holder by providing a spring or flex action to tighten around the blade holder, as shown in FIGS. 8A-8C, the blade 140 is shown operably coupled with the blade holder 150 and the circular end portion 144 and the stepped middle portion 146 sits and seals with the inner section 156 and the stepped portion 159 and the second end 157 of the blade holder.

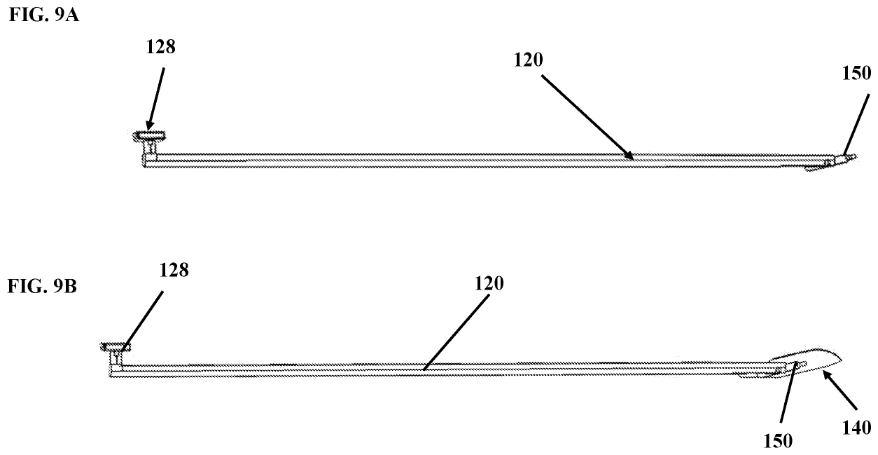

As shown in FIGS. 9A-9B, the button driver 120 is operably coupled with the blade holder 150 and the blade 140.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A surgical blade handle assembly comprising:
a handle operably coupled with a button driver and a proximal end of a shaft;
a blade disposed on the distal end of the shaft and the blade operably coupled with the button driver to protrude out of the inner pocket of the shaft;
the surgical blade handle assembly includes a horizontal axis and a vertical axis, where the button driver operates to displace the blade along the vertical and horizontal axis;
the button driver operates from a first position to a second position as to displace the blade and protrude the blade from the outer surface of the shaft; and
the shaft operates to conceal the blade from the outer portion of the shaft and to not cut any tissue once the blade is in the first position; wherein the second position of the blade is in a cutting position and protrudes out of the outer portion of the shaft.

2. The surgical blade handle assembly of claim 1, wherein the button driver comprises a proximal end and a distal end; wherein the proximal end includes a raised button portion that protrudes out of the handle; the distal end includes a holding portion to secure a blade holder; the blade holder secures the blade to the button driver and is operably coupled with the shaft to displace the blade within a lumen of the shaft and the outer portion of the shaft; and the blade is displaced or protrudes outside the outer portion of the shaft by a distance Db.

3. The surgical blade handle assembly of claim 2, wherein the holding portion includes a first arm and a second arm with a first slotted portion disposed between the first arm and the second arm; the first arm includes a first C-shaped receiver end and the second arm includes a second C-shaped receiver end; the button driver includes a button portion that includes a raised feature protruding vertically out of the handle at a distance Da; the button includes a grated surface to enhance gripping by the user or operator; the first and second C-shaped receiver ends and include a width We and a height Hc that receives the holding arms of the blade holder and allows the holding arms of the blade holder to rotate about an axis.

4. The surgical blade handle assembly of claim 3, wherein the blade holder includes a first receiving arm and a second receiving arm, wherein the first and second receiving arms and protrude from an inner section of the blade holder; the inner section includes a first end and a second end separated by a distance Dc; the inner section includes a stepped portion that includes rectangular profile as to secure a portion of the blade; the inner section also includes a distal slopped portion to permit the blade holder to rotate along the slopped edge of the inner pocket; the stepped portion includes two tabs as to lock the blade holder into the first position and the second position; the inner section includes a width Wb and the tabs include a thickness T1 that the less than the width Wb of the inner section; the first and second C-shaped receiver ends and receive and hold the first receiving arm and second receiving arm of the blade holder and allow the blade holder to be displaced along the vertical axis of the surgical blade handle assembly; and the second receiving arm operably coupled with the inner pocket of the shaft.

5. The surgical blade handle assembly of claim 4, wherein the inner pocket of the shaft includes a slopped edge connecting a flat edge; the slopped edge includes a second stop and the flat edge includes a first stop; and the slopped edge includes a third stop; the second stop abuts the second end of the inner section of the blade holder as to prevent the blade holder from moving any further along the vertical or horizontal axis; the third stop abuts with the two tabs of the stepped portion as to prevent the blade holder from moving any further along the slopped edge; the slopped edge operably couples with the second receiving arm of the blade holder to displace the blade holder along the vertical axis upon proximal displacement of the blade holder along the horizontal axis; the first stop abuts the first end of the of the blade holder to prevent further distal movement of the blade holder along the horizontal axis; the slopped edge includes an angle As and the slopped edge includes a height Hs, where the height Hs is the limiting distance the blade holder and blade is able to be displaced vertically along the vertical axis by way of the second receiving arm.

6. The surgical blade handle assembly of claim 5, wherein the inner pocket includes a distance D1 that accommodates the blade holder in the first position and the distance Dc of the blade holder; the inner pocket includes a distance D2 that is greater than D1 and Dc to permit the horizontal displacement of the blade holder within the inner pocket; the inner pocket includes a lower grooved portion to allow the smooth horizontal displacement of the blade holder within the inner pocket.

7. The surgical blade handle assembly of claim 6, wherein the button driver is displaced proximally along the longitudinal axis and moving the blade holder and while the blade holder is proximally displaced, the second receiving arm moves at the Angle A and vertically displaces the blade holder and blade; the first receiving arm allows the blade holder to rotate along the vertical axis; the second receiving arm moves along the angled edge of the inner pocket; the first position includes the blade disposed within the lumen of the shaft and concealed within the shaft.

8. The surgical blade handle assembly of claim 7, wherein the blade includes an inner cutout configurable to fit over the at least a portion of the inner section of the blade holder; the inner cutout includes an opening, a circular end portion, a stepped middle portion, and a second end cap; the stepped portion includes a complementary and reciprocal circular end portion, a stepped middle portion, and a second end cap as to receive the inner cutout of the blade; and the blade includes a sharpened portion.

9. The surgical blade handle assembly of claim 8, wherein the inner cutout incudes a first lipped portion between circular end portion and the stepped middle portion; and a second lipped portion between the stepped middle portion and the second end cap; the first lipped portion includes an angle A1 and the second lipped portion includes an angle A1; the first and second lipped portions assist in securing the blade to the blade holder by providing a spring or flex action to tighten around the blade holder; the blade operably coupled with the blade holder and the circular end portion and the stepped middle portion sits and seals with the inner section and the stepped portion and the second end of the blade holder.

10. A method of operating a surgical blade handle assembly comprising:
Coupling a handle operably with a button driver and a proximal end of a shaft, and the surgical blade handle assembly includes a horizontal axis and a vertical axis;
Disposing a blade on the distal end of the shaft and operably coupling the blade with the button driver to protrude out of the inner pocket of the shaft;
Operating the button driver to displace the blade along the vertical and horizontal axis;
Operating the button driver from a first position to a second position as to displace the blade and protrude the blade from the outer surface of the shaft; and
Operating the shaft to conceal the blade from the outer portion of the shaft and to not cut any tissue once the blade is in the first position; wherein the second position of the blade is in a cutting position and protrudes out of the outer portion of the shaft.

11. The method of claim 10, wherein the button driver comprises a proximal end and a distal end; including a raised button portion on the proximal end that protrudes out of the handle; including a holding portion on the distal to secure a blade holder; securing the blade to the button driver by the blade holder and operably coupling the button driver with the shaft to displace the blade within a lumen of the shaft and the outer portion of the shaft; and displacing the blade outside the outer portion of the shaft by a distance Db.

12. The method of claim 11, including a first arm and a second arm with the holding portion and the second arm with a first slotted portion disposed between the first arm and the second arm; including a first C-shaped receiver end with the first arm and the second arm includes a second C-shaped receiver end; including a button portion with the button driver that includes a raised feature protruding vertically out of the handle at a distance Da; including a grated surface with the button to enhance gripping by the user or operator; allowing the holding arms of the blade holder to rotate about an axis with the first and second C-shaped receiver ends including a width We and a height Hc that receives the holding arms of the blade holder.

13. The method of claim 12, including a first receiving arm and a second receiving arm with the blade holder, and protruding the first and second receiving arms from an inner section of the blade holder; including a first end and a second end with the inner section separated by a distance Dc; including a stepped portion with the inner section that includes rectangular profile as to secure a portion of the blade; permitting the blade holder to rotate along the slopped edge of the inner pocket with the inner section including a distal slopped portion to; locking the blade holder into the first position and the second position the stepped portion including two tabs; including a width Wb with the inner section and the tabs include a thickness T1 that the less than the width Wb of the inner section; receiving and holding the first receiving arm and the second receiving arm of the blade holder with the first and second C-shaped receiver ends, and allowing the blade holder to be displaced along the vertical axis of the surgical blade handle assembly; and operably coupling the second receiving arm with the inner pocket of the shaft.

14. The method of claim 13, including a slopped edge with the inner pocket of the shaft connecting a flat edge; including a second stop with the slopped edge and the flat edge includes a first stop; and the slopped edge includes a third stop; abutting the second end of the inner section of the blade holder with the second stop and preventing the blade holder from moving any further along the vertical or horizontal axis; abutting the two tabs of the stepped portion with the third stop and preventing the blade holder from moving any further along the slopped edge; operably coupling the slopped edge with the second receiving arm of the blade holder to displace the blade holder along the vertical axis upon proximal displacement of the blade holder along the horizontal axis; abutting the first stop with the first end of the of the blade holder and preventing further distal movement of the blade holder along the horizontal axis; including an angle As with the slopped edge and the slopped edge includes a height Hs, where the height Hs is the limiting distance the blade holder and displacing the blade vertically along the vertical axis by way of the second receiving arm.

15. The method of claim 14, including a distance D1 with the inner pocket and accommodating the blade holder in the first position and the distance Dc of the blade holder; including a distance D2 with the inner pocket that is greater than D1 and Dc and permitting the horizontal displacement of the blade holder within the inner pocket; including a lower grooved portion with the inner pocket and allowing the smooth horizontal displacement of the blade holder within the inner pocket.

16. The method of claim 15, displacing the button driver proximally along the longitudinal axis and moving the blade holder and while the blade holder is proximally displaced, moving the second receiving arm at the Angle A and vertically displacing the blade holder and blade; allowing the blade holder to rotate along the vertical axis by the first receiving arm; moving the second receiving arm along the angled edge of the inner pocket; disposing the blade in the first position within the lumen of the shaft and concealed within the shaft.

17. The method of claim 16, including an inner cutout with the blade configurable to fit over the at least a portion of the inner section of the blade holder; including an opening, a circular end portion, a stepped middle portion, and a second end cap with the inner cutout; including a complementary and reciprocal circular end portion, a stepped middle portion, and a second end cap with the stepped portion as to receive the inner cutout of the blade; and including a sharpened portion with the blade.

18. The method of claim 17, including a first lipped portion with the inner cutout between circular end portion and the stepped middle portion; and a second lipped portion between the stepped middle portion and the second end cap; including an angle A1 with the first lipped portion and including an angle A1 with the second lipped portion; securing the blade to the blade holder with the first and second lipped portions assisting by providing a spring or flex action to tighten around the blade holder; operably coupling the blade with the blade holder and the circular end portion and sitting the stepped middle portion and sealing with the inner section and the stepped portion and the second end of the blade holder.

19. A surgical blade handle assembly comprising:
a handle operably coupled with a button driver and a proximal end of a shaft;
a blade disposed on the distal end of the shaft and the blade operably coupled with the button driver to protrude out of the inner pocket of the shaft;
the surgical blade handle assembly includes a horizontal axis and a vertical axis, where the button driver operates to displace the blade along the vertical and horizontal axis;
the button driver operates from a first position to a second position as to displace the blade and protrude the blade from the outer surface of the shaft;
the shaft operates to conceal the blade from the outer portion of the shaft and to not cut any tissue once the blade is in the first position; wherein the second position of the blade is in a cutting position and protrudes out of the outer portion of the shaft;
wherein the button driver comprises a proximal end and a distal end; wherein the proximal end includes a raised button portion that protrudes out of the handle; the distal end includes a holding portion to secure a blade holder; the blade holder secures the blade to the button driver and is operably coupled with the shaft to displace the blade within a lumen of the shaft and the outer portion of the shaft; and the blade is displaced or protrudes outside the outer portion of the shaft by a distance Db;
wherein the holding portion includes a first arm and a second arm with a first slotted portion disposed between the first arm and the second arm; the first arm includes a first C-shaped receiver end and the second arm includes a second C-shaped receiver end; the button driver includes a button portion that includes a raised feature protruding vertically out of the handle at a distance Da; the button includes a grated surface to enhance gripping by the user or operator; the first and second C-shaped receiver ends and include a width We and a height Hc that receives the holding arms of the blade holder and allows the holding arms of the blade holder to rotate about an axis;
wherein the blade holder includes a first receiving arm and a second receiving arm, wherein the first and second receiving arms and protrude from an inner section of the blade holder; the inner section includes a first end and a second end separated by a distance Dc; the inner section includes a stepped portion that includes rectangular profile as to secure a portion of the blade; the inner section also includes a distal slopped portion to permit the blade holder to rotate along the slopped edge of the inner pocket; the stepped portion includes two tabs as to lock the blade holder into the first position and the second position; the inner section includes a width Wb and the tabs include a thickness T1 that the less than the width Wb of the inner section; the first and second C-shaped receiver ends and receive and hold the first receiving arm and second receiving arm of the blade holder and allow the blade holder to be displaced along the vertical axis of the surgical blade handle assembly; and the second receiving arm operably coupled with the inner pocket of the shaft; and
wherein the inner pocket of the shaft includes a slopped edge connecting a flat edge; the slopped edge includes a second stop and the flat edge includes a first stop; and the slopped edge includes a third stop; the second stop abuts the second end of the inner section of the blade holder as to prevent the blade holder from moving any further along the vertical or horizontal axis; the third stop abuts with the two tabs of the stepped portion as to prevent the blade holder from moving any further along the slopped edge; the slopped edge operably couples with the second receiving arm of the blade holder to displace the blade holder along the vertical axis upon proximal displacement of the blade holder along the horizontal axis; the first stop abuts the first end of the of the blade holder to prevent further distal movement of the blade holder along the horizontal axis; the slopped edge includes an angle As and the slopped edge includes a height Hs, where the height Hs is the limiting distance the blade holder and blade is able to be displaced vertically along the vertical axis by way of the second receiving arm.

20. The surgical blade handle assembly of claim 19, wherein the inner pocket includes a distance D1 that accommodates the blade holder in the first position and the distance Dc of the blade holder; the inner pocket includes a distance D2 that is greater than D1 and Dc to permit the horizontal displacement of the blade holder within the inner pocket; the inner pocket includes a lower grooved portion to allow the smooth horizontal displacement of the blade holder within the inner pocket.
